(19) United States
(12) Patent Application Publication
(10) Pub. No.: US 2008/0102130 A1
(43) Pub. Date: May 1, 2008

(54) DRY GRANULATED PHARMACEUTICAL COMPOSITIONS AND METHODS FOR PRODUCING SAME

(75) Inventors: Abraham B. Woldu, Souderton, PA (US); John Cunningham, Lansdale, PA (US)

Correspondence Address: WOODCOCK WASHBURN LLP, CIRA CENTRE, 12TH FLOOR, 2929 ARCH STREET, PHILADELPHIA, PA, 19104-2891, US

(73) Assignee: Janssen Pharmaceutica NV, Beerse (BE)

(21) Appl. No.: 11/925,614

(22) Filed: Oct. 26, 2007

Related U.S. Application Data
(60) Provisional application No. 60/863,317, filed on Oct. 27, 2006.

(51) Int. Cl. A61K 9/16

(52) U.S. Cl. 424/489

(57) ABSTRACT

Method are provided for dry granulation processing of a pharmaceutical composition to provide granulated pharmaceutical compositions with improved flow characteristics and reduced amounts of fine particles.

### REFERENCE TO RELATED APPLICATION

This application claims benefit under 35 U.S.C. § 119(e) of U.S. Application No. 60/863,317 filed Oct. 27, 2006, the entirety of which is incorporated herein by reference.

### FIELD

The invention relates generally to methods for dry granulation processing pharmaceutical compositions and, in particular, to granulated pharmaceutical compositions with improved flow characteristics and a reduced amount of fine particles.

### BACKGROUND

Dry granulation processes provide viable options for poor-flowing, moisture-sensitive compounds. However, a need exists in the art to develop an improved dry granulation process that provides a drug composition that is a flowable final blend containing a relatively low amount of fine particles.

### SUMMARY

The present invention provides methods for dry granulation processing of a pharmaceutical composition to provide a composition with improved flow characteristics and a reduced amount of fine particles. Preferred methods comprise compressing a pharmaceutical composition to a predetermined hardness (preferably about 800-900 kPa) to produce one or more slugs, and milling the slug(s) with an oscillating granulator to form granules. The granules thus produced can then be sized, for example, with a sieve within the oscillating granulator. The oscillating granulator can be a Stokes oscillating granulator. The oscillating granulator can have a 0.25 inch screen for milling the slugs and a 16 mesh screen for sizing the granules. The sized granules can have an average diameter from about 100 to about 200 microns. In a detailed aspect, the sized granules can have an average diameter of about 150 microns. In a further detailed aspect, no more than 35% of the sized granules have a diameter that is about 75 microns or less.

### BRIEF DESCRIPTION OF THE DRAWINGS

FIGS. 1A, 1B, and 1C shows experimental design for slug milling equipment, slug hardness, and final milling equipment.

FIG. 2 shows the effect of milling equipment on slug milling as measured by sieve analysis through a 20 mesh screen.

FIG. 3 shows the effect of slug hardness as measured by sieve analysis of the final preblend granulation.

FIG. 4 shows the effect of the Comil, Fitzmill, and Oscillator on final milling as measured by sieve analysis of the final preblend granulation.

FIG. 5 shows the effect of the Oscillator on slug milling as measured by sieve analysis of the final preblend granulation.

FIG. 6 shows the effect of the Fitzmill on slug milling as measured by sieve analysis of the final preblend granulation.

FIG. 7 shows the effect of compression force on tablet hardness for Comil-Comil, Comil-Fitzmill, or Comil-Oscillator milling.

FIG. 8 shows the effect of compression force on tablet hardness for Comil-Comil, and Oscillator-Oscillator or Fitzmill-Fitzmill milling.

FIG. 9 shows the effect of compression force on tablet hardness for Comil-Comil, milling at 6 kp, 8 kp, or 10 kp.

FIG. 10 shows dissolution rates of pharmaceutical compositions milled by an Oscillator milling-Oscillator sieving process.

### DETAILED DESCRIPTION

The present invention provides methods for dry granulation processing of a pharmaceutical composition to improve flow of the pharmaceutical composition. Such methods are believed to be applicable to any composition that includes at least one active pharmaceutical ingredient (API). Particularly preferred APIs are those that are moisture and/or heat sensitive, and hence cannot be wet granulated, and those APIs which have batch to batch variation in morphology, mean particle size, particle size distribution, density, electrostatic nature and other bulk properties that result in poor and variable flow, or distribution of the API in the final blend. Pharmaceutical compositions according the invention can also include one or more carrier, excipient, diluent, stabilizer, buffer or other pharmaceutically acceptable additives, such as ascorbic acid or glutathione, chelating agents, low molecular weight proteins, compositions that reduce the clearance or hydrolysis of the pharmaceutical formulation. Representative APIs and additives are known to the skilled artisan and are described in detail in the scientific and patent literature, e.g., Remington's Pharmaceutical Science, 18^{th} Edition, 1990, Mack Publishing Company, Easton, Pa. (“Remington's”), or Physicians Desk Reference, Thompson, 2006. The methods of the invention are believed to be particularly useful for processing moisture sensitive compositions.

The methods of the invention involve compressing a pharmaceutical composition, preferably to a hardness of from about 7 kiloponds to about 9 kiloponds, preferably about 8 kiloponds, to produce one or more slugs. The hardness measurement is equivalent to about 600 kPa to about 1100 kPa, or preferably about 800 kPa. Hardness test is intended to determine, under defined conditions, the resistance to crushing of slugs, granules or tablets, measured by the force needed to disrupt them by crushing. The results are usually expressed in Newton or kiloponds. In this work 8M tablet-Hardness Testing machine, Dr. Scheuniger Pharmatron with S.N. 02228 was used. The description of the technique can be found on European Pharmacopeia-2006 (01/2005:20909) Any of the many types of devices known in the art can be used to compress the composition and produce the slugs. Preferred devices include, for example, rotary tablet presses which have a system of monitoring and controlling the compression profile including, but not limited to, the Manesty brand Betapress and SMI Directory System, model V3.0200 (SMI Inc.).

The methods of the invention also involve milling the slugs to form granules. Although any of the many types of milling devices known in the art can be used, it is preferred that an oscillating granulator can be used, for example, a Stokes oscillating granulator or an oscillating granulator from another manufacturer. The granulator preferably produces granules having a mean particle size of about 100 micron to about 200 micron (preferably about 150 micron). In embodiments of the invention in which an oscillating granulator is used, it preferably is equipped with a screen (preferably a 0.25 inch screen) for milling the slugs.

Once formed, granules according to the invention are sized, i.e., granules of desirable size(s) or falling within a one or more desirable size ranges are separated from granules of undesirable size(s) or falling within a one or more undesirable size ranges. Any of the many sizing techniques and devices known in the art can be used, although it is preferred to use an oscillating granulator that is equipped with a screen (preferably a 16 mesh screen) suitably positioned to size the granules that are produced in the milling step. Sized granules preferably have an average diameter from about 150 to about 200 microns (more preferably about 170 microns) and/or no more than about 35% of the sized granules have a diameter that is about 75 microns or less.

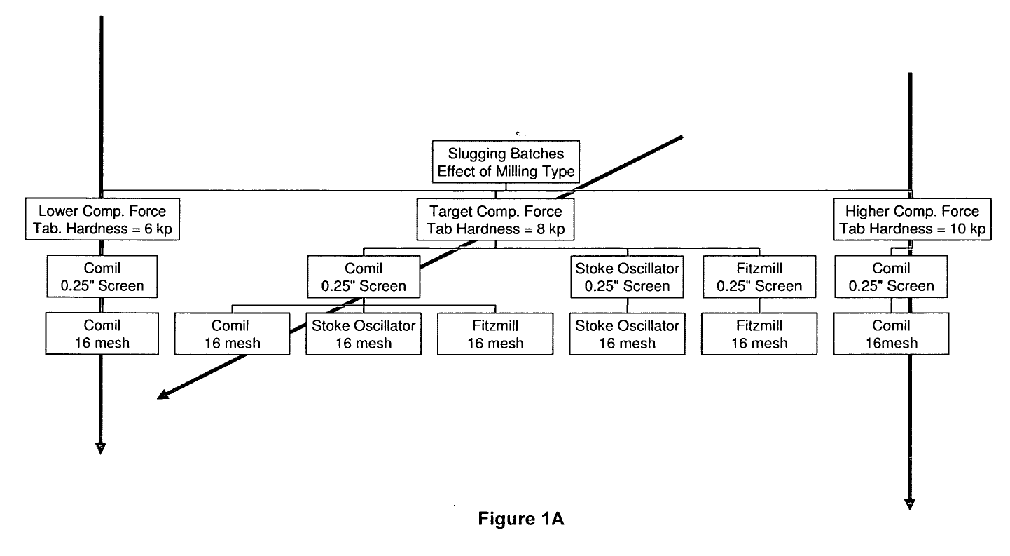

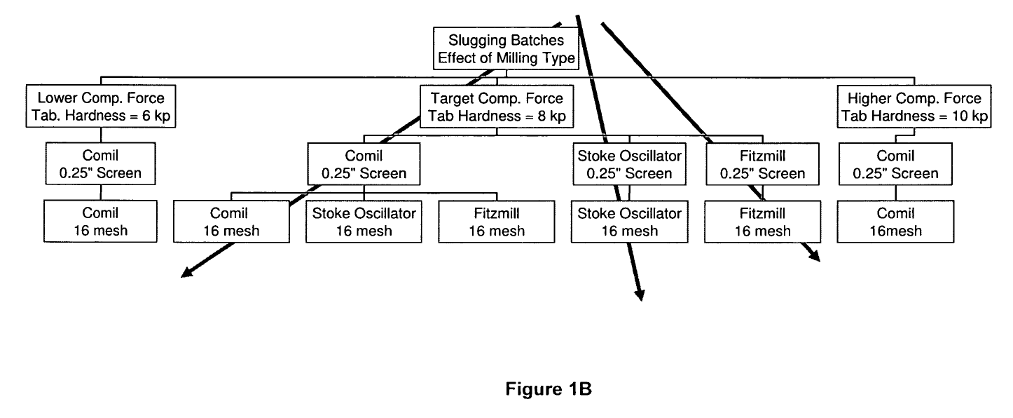

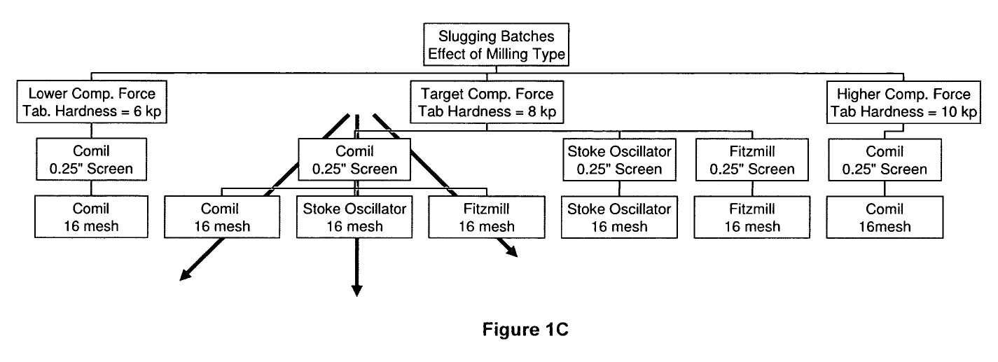

In one representative embodiment of the present invention, an exemplary pharmaceutical formulation as in Table 1 containing an active pharmaceutical ingredient was processed to flat round 14.3 mm slugs that were compressed to 6 kilo-ponds (kp), 8 kp, and 10 kp hardness on a Manesty Betapress. 626 kPa is equivalent to 6 kp, 833 kPa is equivalent to 8 kp, and 1041 kPa (1.041 MPa) is equivalent to 10 kp. The slugs were further processed via the Quadro Comil 197s with round impeller and either no spacers for slug milling or 0.125 inches of spacers for final milling; Stokes Oscillator 43A; and/or Fitzmill Homoloid JT6 equipped with 6.35 mm (0.25 inch) screen for initial milling and 1.18 mm (16 mesh) screen for final sizing. FIGS. 1A, 1B, and 1C summarize the milling method design of the experiment.

Tapentadol is an API which is a highly water soluble centrally acting analgesic. Tapentadol is predominantly rectangular or rod-shaped crystalline powder. The particle size distribution of the drug substance used in this work had a range of D50 from 50 to 250 microns. The D10 can be as low as 5 microns while the D99 can be as high as 500 microns. Although, the particle size distribution of the drug substance is controlled during crystallization, milling or micronizing the API to less than a micron size would not affect processability, as described herein, or the attribute of the drug product. Other APIs with similar properties such as Tramadol are expected to have a similar behavior.

TABLE 1
Exemplary Pharmaceutical Formulation for
an Active Pharmaceutical Ingredient (API)

| Raw Materials | Target % w/w | Range (% w/w) |
|---|---|---|
| Tapentadol HCl | 33 | 8-50 |
| Hypromellose 2208 | 14 | 10-40 |
| Microcrystalline Cellulose | 51 | 20-80 |
| Colloidal Silicon Dioxide | 0.5 | 0.10-0.75 |
| Magnesium Stearate | 0.3 | 0.10-0.75 |
| Extra-granular additions | | |
| Colloidal Silicon Dioxide | 0.5 | 0.10-0.75 |
| Magnesium Stearate | 0.3 | 0.10-0.75 |
| Totals | 100 | 100 |

Factors to consider in milling method design include slug hardness (compression force), and milling techniques for first pass milling and final milling. Properties of the dry granulation process were measured as particle size distribution, density, flow testing, compression profile and tablet properties.

Particle size analysis post initial milling of the slugs, was measured by the percent of slugged milled material that passed through a 20 mesh screen.

Bulk and tap densities, particle size, and flow analysis were obtained for the final milled blended material. The Sotax Flow Tester produced the flow data, where the flow-rate of the sample was measured as a ratio (α/α_{ref}) to that of reference (granular sand).

FIGS. 1A, 1B, and 1C show experimental design for slug milling equipment, slug hardness, and final milling equipment. FIG. 1A shows a slugging batch process varying target compression force tab hardness of 6 kp, 8 kp, or 10 kp, with a Comil 0.25 inch screen for initial milling and Comil 16 mesh for final sizing. 626 kPa is equivalent to 6 kp, 833 kPa is equivalent to 8 kp, and 1041 kPa (1.041 MPa) is equivalent to 10 kp. FIG. 1B shows a slugging batch process varying slug milling equipment using target compression force tab hardness of 8 kp, with a Comil 0.25 inch screen for initial milling and Comil 16 mesh for final sizing; a Stokes Oscillator 0.25 inch screen for initial milling and Stoke Oscillator 16 mesh for final sizing; or a Fitzmill 0.25 inch screen for initial milling and Fitzmill 16 mesh for final sizing. FIG. 1C shows a slugging batch process varying final milling equipment using target compression force tab hardness of 8 kp, with a Comil 0.25 inch screen for initial milling and Comil 16 mesh for final sizing; with a Comil 0.25 inch screen for initial milling and Stoke Oscillator 16 mesh for final sizing; or with a Comil 0.25 inch screen for initial milling and Fitzmill 16 mesh for final sizing.

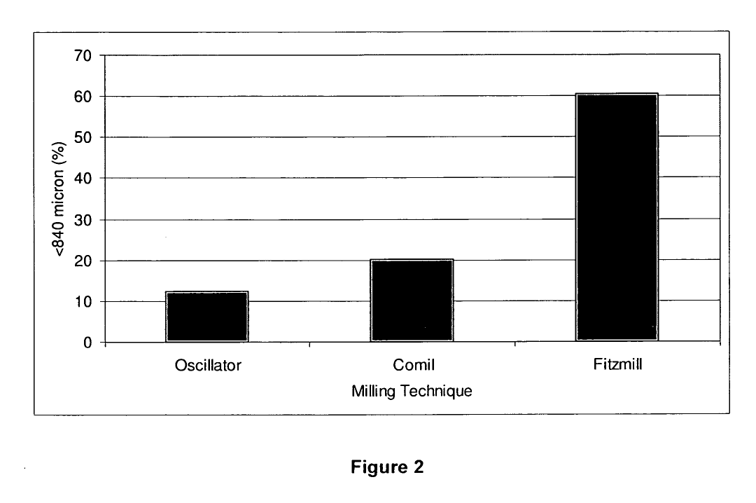

FIG. 2 shows the effect of milling equipment on slug milling as measured by sieve analysis through a 20 mesh screen. The Stokes Oscillator produces the lowest percentage of fine particles less than 840 microns.

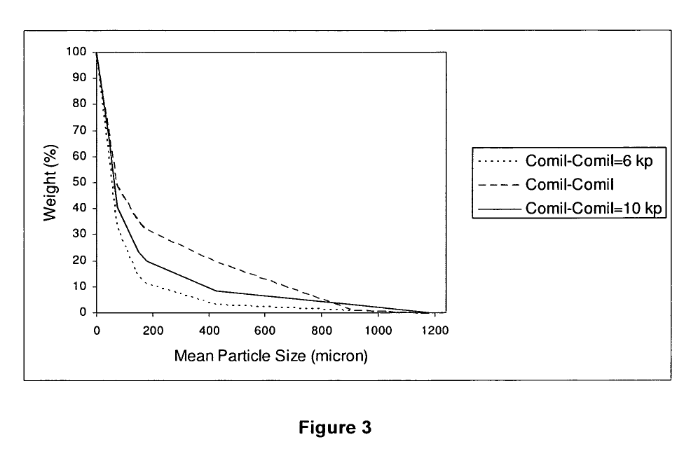

FIG. 3 shows effect of slug hardness as measured by sieve analysis of the final preblend granulation. The figure shows that slug hardness of 8 kp with a Comil 0.25 inch screen and Comil 16 mesh process provides a larger mean particle size following final preblend granulation.

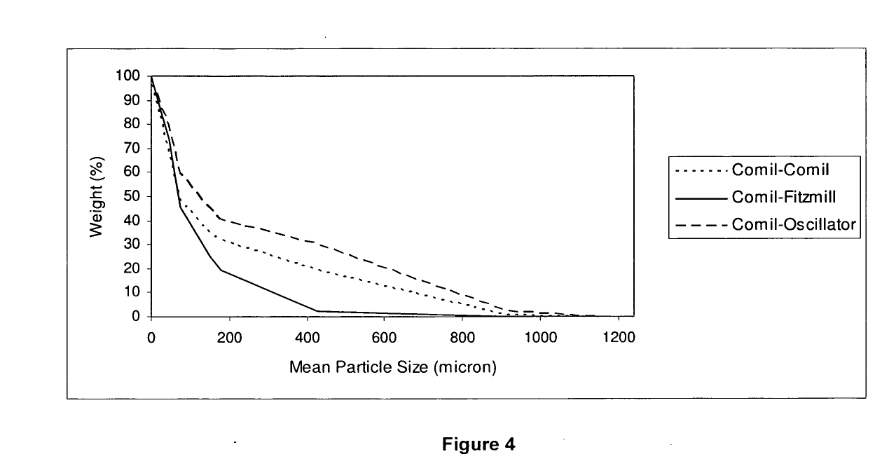

FIG. 4 shows effects of the Comil, Fitzmill, and Oscillator on final milling as measured by sieve analysis of the final preblend granulation. The figure shows that slug hardness of 8 kp with a Comil 0.25 inch screen and Oscillator 16 mesh process provides a larger mean particle size following final preblend granulation.

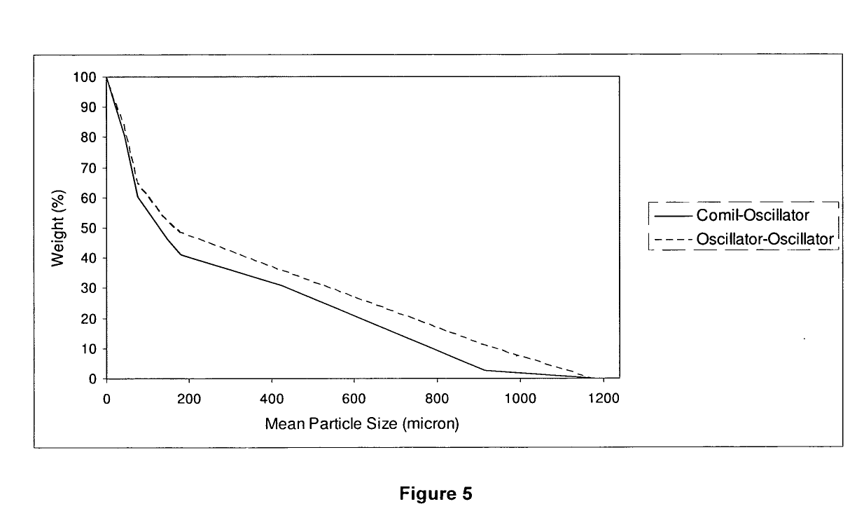

FIG. 5 shows effects of the Oscillator on slug milling as measured by sieve analysis of the final preblend granulation. The figure shows that slug hardness of 8 kp with a with an Oscillator 0.25 inch screen and Oscillator 16 mesh process provides a larger mean particle size following final preblend granulation.

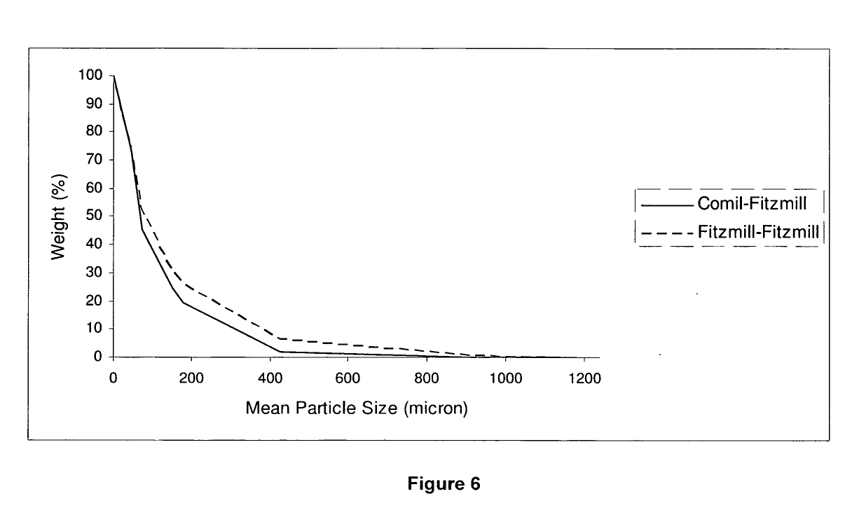

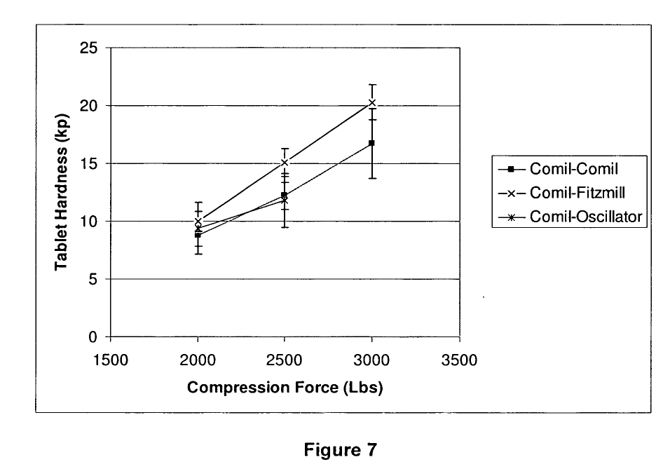

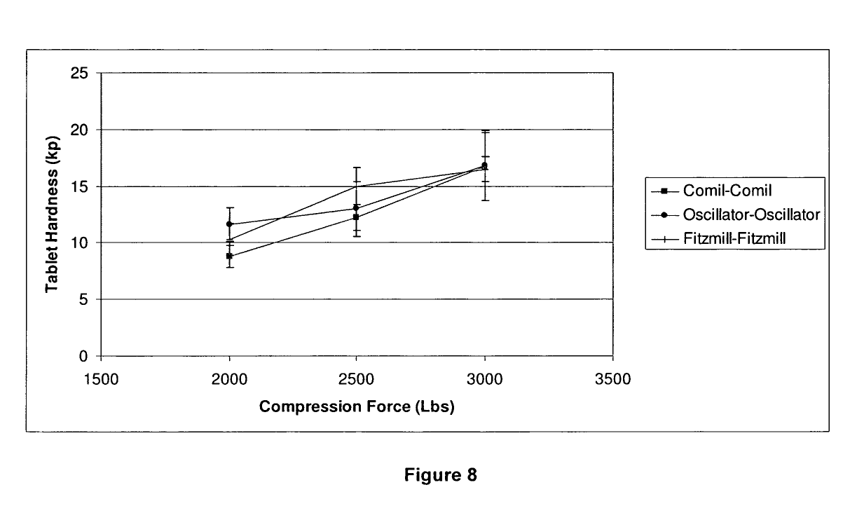

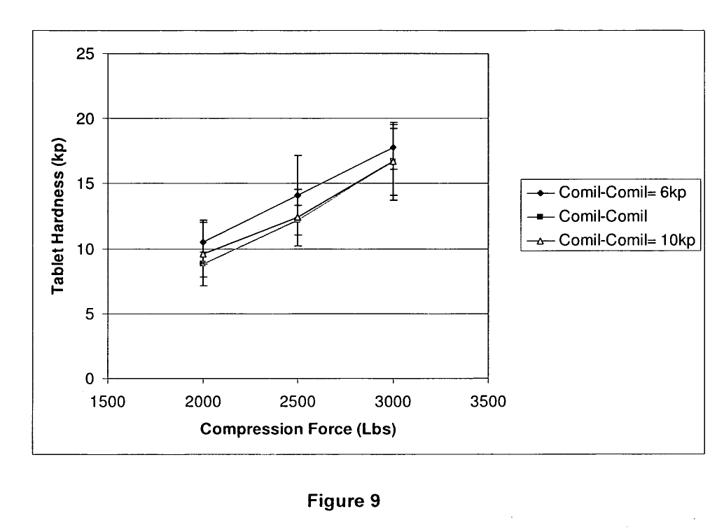

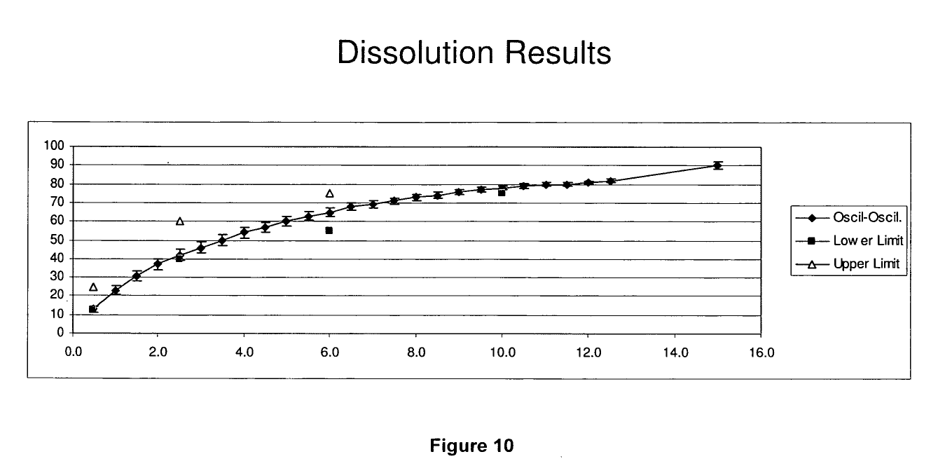

FIG. 6 shows effects of the Fitzmill on slug milling as measured by sieve analysis of the final preblend granulation. The figure shows that slug hardness of 8 kp with a Fitzmill 0.25 inch screen and Fitzmill 16 mesh process provides a slightly larger mean particle size than a Comil 0.25 inch screen and Fitzmill 16 mesh following final preblend granulation.

Table 2 shows the effect of slug hardness on the physical characteristics of the final blend. The table shows that a slug hardness of 8 kp with a Comil 0.25 inch screen and Comil 16 mesh process provides a mean particle size of 75 microns and about 50.5% of the particles are less than 75 micron.

TABLE 2
Effect of slug hardness on the physical
characteristics of the final blend.

| | Comil-Comil = | Comil-Comil = | Comil-Comil = |
|---|---|---|---|
| | 6 kp | 8 kp | 10 kp |
| Flow (Sotax-ratio) | | | |
| Post Final Miling | 0.31 | 0.24 | 0.2 |
| Final Blend | 0.3 | 0.34 | 0.29 |
| Density | | | |
| Bulk Density (g/mL) | 0.45 | 0.47 | 0.45 |
| Tap Density (g/mL) | 0.68 | 0.74 | 0.71 |
| Particle Size | | | |
| D50 (microns) | 56 | 75 | 64 |
| <75 micron (%) | 66.5 | 50.5 | 59.9 |

Table 3 shows the effect of milling equipment on the physical characteristics of the final blend. The table shows that a slug hardness of 8 kp with an Oscillator 0.25 inch screen and an Oscillator 16 mesh process provides a mean particle size of about 172 microns and about 34.5% of the particles are less than 75 micron.

TABLE 3
Effect of milling equipment on the physical
characteristics of the final blend.

| | | | Oscillator- |
|---|---|---|---|
| | Comil-Comil | Fitzmill-Fitzmill | Oscill. |
| Flow (Sotax-ratio) | | | |
| Post Final Miling | 0.31 | 0.39 | 0.4 |
| Final Blend | 0.3 | 0.55 | 0.55 |
| Density | | | |
| Bulk Density (g/mL) | 0.45 | 0.48 | 0.45 |
| Tap Density (g/mL) | 0.68 | 0.69 | 0.71 |
| Particle Size | | | |
| D50 (microns) | 56 | 85 | 172 |
| <75 micron | 66.5 | 47 | 34.5 |

Table 4 shows a summary of the experimental results discussed above. Round flat slugs with 14.3 mm diameter were compressed to 6 kilo-ponds (kp), 8 kp, and 10 kp hardness. 626 kPa is equivalent to 6 kp, 833 kPa is equivalent to 8 kp, and 1041 kPa (1.041 MPa) is equivalent to 10 kp. The mills utilized were the Quadro Comil 197s, Stokes Oscillator 43A, and Fitzmill Homoloid JT6 equipped with 6.35 mm screen for initial milling and 1.18 mm screen for final sizing. Bulk and Tap densities, particle size, and flow test were obtained using the Sotax Flow Tester, where the flow-rate of the sample is obtained as a ratio (α/α_{ref}) to that of a reference (granular sand).

The results indicate the mid-point hardness of 8 kp yields the most desirable final blend flow, the least amount of fines, a larger mean particle size, and a denser granulation.

TABLE 4
Effects of Slug hardness on the properties of the granules

| Slug | Fines | Median | Relative | | |
|---|---|---|---|---|---|
| Hardness | (<75 μm) | Particle | flow ratio | Density (g/mL) | |
| (kp) | (%) | size D₅₀ (μm) | (α/α_{ref}) | Bulk | Tap |
| 6 | 66.5 | 56 | 0.3 | 0.45 | 0.68 |
| 8 | 50.5 | 75 | 0.34 | 0.47 | 0.74 |
| 10 | 59.9 | 64 | 0.29 | 0.45 | 0.71 |

Comparison among the different milling techniques indicated that the Stokes Oscillator and Fitzmill Holomoid produce similar final blend flow (α/α_{ref}=0.55)while that of the Comil was considerably lower (α/α_{ref}=0.30). The Stokes Oscillator produced granules of the largest mean particle size with the least amount of particles under 75 microns (Stokes: D₅₀=172 micron and 34.5%<75 microns; Fitzmill: D₅₀=85 micron and 37.0%<75 microns; Comil: D₅₀=56 micron and 66.5%<75 microns).

The studies suggest the most flowable final blend containing the least amount of fine particle for this formulation is achieved by utilizing the Stokes Oscillating Granulator and an initial slug hardness of 8 kp.

In exemplary formulations, the ratio of active pharmaceutical ingredient (API) to microcrystalline cellulose (MCC) ranged from a ratio of 1:10 to 5:2. The ratio of the API to Hypromellose ranged from 1:5 to 5:1. The ratio of MCC to Hypromellose ranged from 1:2 to 8:1. The slugging process includes, but not limited to, the following steps:
1. Screen API, Metolose and MCC and Colloidal silicon dioxide through #20 mesh.
2. Transfer the screened materials into the Bohle Bin Blender 20 L and blend for 10 minutes at the speed of 25 rpm.
3. Screen the magnesium stearate through a #30 mesh and load into the blender in step 2 and blend for 5 minutes at the speed 25 rpm.
4. Sample the blended materials for flowability, moisture, bulk and tap densities, and particle size distribution analysis.
5. Transfer the material for compression on the Manesty Betapress. Use 16 stations and round and flat shaped tooling with diameter range of 14 to 20 mm (preferably 20 mm). Compress the slugs to a specified hardness.
6. Use Stoke Oscillator fitted with a 3 mesh screen to mill (Fist-Phase) if not specified in the given batch record
7. Screen the granules from Step 6 and colloidal silicon dioxide (extra-granular) through #20 mesh. Load the screened materials into 20 L Bohle Bin Blender. Blend the materials for 5 minutes and at the speed of 25 rpm.
8. Screen the magnesium stearate through a #30 mesh. Load the material into the blender from the previous step and blend for 5 min.
9. Samples were taken from the final blend for flowability, moisture, bulk and tap densities, and particle size distribution analysis.
10. Transfer the material for compression.
11. Compress the material using the Manesty Betapress with 16 stations using 17×7mm modified capsule shaped tooling.

The mechanical strength of a pharmaceutical powder compact is a complex function of the properties of the materials, which constitute the compact and the dynamic process stress to which the individual particles are subjected. Thus it is important to select a procedure that results in compacts of required properties. It is also important to identify a standard procedure that enables one to indicate the mechanical strength of the compact. Due to their brittle nature, pharmaceutical compacts usually fail in tension during stress. Tensile strength is the property of a compact to resist failure from tensile stress. This technique does not depend on the slug or tablet thickness. Characterization of pharmaceutical compacts is achieved by the application of diametral compression (J. T. Fell and J. M. Newton, “Determination of tablet strength by the diametral-compression test,” J. Pharm. Sci. 59: 688-691, 1970).
σ=2P/πDT

Where σ is the tensile strength (Pa), P is the breaking force (N), D is the tablet diameter (m) and Tis the thickness of tablet (m). (Fell and Newton, 1970)

The slugs were compressed to have 626 kPa (equivalent to 6 kp), 833 kPa (equivalent to 8 kp) and 1041 kPa (1.041 MPa) (equivalent to 10 kp). Slugs were produced with approximately 1000 mg weight (800-1500 mg). The thickness of the slugs varies inversely with the diameter. The range of the slug diameter in this project was from 14 mm to 20 mm with respective approximate slug thickness of 6 mm and 3 mm. Tablets with different hardness resulted, typically in the range of from 800 to 900 kPa.

Pharmaceutical compositions according to the invention can be incorporated into liquid or solid pharmaceutical formulations. Representative liquid formulations are those in which the pharmaceutical composition is dissolved in a pharmaceutically acceptable carrier, e.g., an aqueous carrier if the composition is water-soluble. Examples of aqueous solutions that can be used in formulations for enteral, parenteral or transmucosal drug delivery include, e.g., water, saline, phosphate buffered saline, Hank's solution, Ringer's solution, dextrose/saline, glucose solutions and the like. The formulations can contain pharmaceutically acceptable auxiliary substances as required to approximate physiological conditions, such as buffering agents, tonicity adjusting agents, wetting agents, detergents and the like. Additives can also include additional active ingredients such as bactericidal agents, or stabilizers. For example, the solution can contain sodium acetate, sodium lactate, sodium chloride, potassium chloride, calcium chloride, sorbitan monolaurate or triethanolamine oleate. These compositions can be sterilized by conventional, well-known sterilization techniques, or can be sterile filtered. The resulting aqueous solutions can be packaged for use as is, or lyophilized, the lyophilized preparation being combined with a sterile aqueous solution prior to administration. The concentration of active compound in these formulations can vary widely, and will be selected primarily based on fluid volumes, viscosities, body weight and the like in accordance with the particular mode of administration selected and the patient's needs.

Solid pharmaceutical formulations can be formulated as, e.g., pills, tablets, powders or capsules. For such formulations, conventional nontoxic solid carriers can be used which include, e.g., pharmaceutical grades of mannitol, lactose, starch, magnesium stearate, sodium saccharin, talcum, cellulose, glucose, sucrose, magnesium carbonate, and the like. For oral administration, a pharmaceutically acceptable nontoxic composition is formed by incorporating any of the normally employed excipients, such as those carriers previously listed, and generally 10% to 95% of active ingredient. A non-solid formulation can also be used for enteral administration. The carrier can be selected from various oils including those of petroleum, animal, vegetable or synthetic origin, e.g., peanut oil, soybean oil, mineral oil, sesame oil, and the like. Suitable pharmaceutical excipients include e.g., starch, cellulose, talc, glucose, lactose, sucrose, gelatin, malt, rice, flour, chalk, silica gel, magnesium stearate, sodium stearate, glycerol monostearate, sodium chloride, dried skim milk, glycerol, propylene glycol, water, ethanol.

Pharmaceutical formulations of the invention, when administered orally, can be protected from digestion. This can be accomplished either by complexing the pharmaceutical formulation with a composition to render it resistant to acidic and enzymatic hydrolysis or by packaging the pharmaceutical formulations in an appropriately resistant carrier such as a liposome. Means of protecting compounds from digestion are well known in the art, see, e.g., Fix, Pharm Res. 13: 1760-1764, 1996; Samanen, J. Pharm. Pharmacol. 48: 119-135, 1996; U.S. Pat. No. 5,391,377, describing lipid compositions for oral delivery of therapeutic agents (liposomal delivery is discussed in further detail, infra).

In preparing pharmaceutical formulations of the present invention, a variety of modifications can be used and manipulated to alter pharmacokinetics and biodistribution. A number of methods for altering pharmacokinetics and biodistribution are known to one of ordinary skill in the art. Examples of such methods include protection of the compositions of the invention in vesicles composed of substances such as proteins, lipids (for example, liposomes, see below), carbohydrates, or synthetic polymers (discussed above). For a general discussion of pharmacokinetics, see, e.g., Remington's, Chapters 37-39.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier.

The pharmaceutical compositions are generally formulated as sterile, substantially isotonic and in full compliance with all Good Manufacturing Practice (GMP) regulations of the U.S. Food and Drug Administration.

All publications and patent applications cited in this specification are herein incorporated by reference in their entirety for all purposes as if each individual publication or patent application were specifically and individually indicated to be incorporated by reference for all purposes.

Although the foregoing invention has been described in some detail by way of illustration and example for purposes of clarity of understanding, it will be readily apparent to one of ordinary skill in the art in light of the teachings of this invention that certain changes and modifications may be made thereto without departing from the spirit or scope of the appended claims.

What is claimed:

1. A method for producing a dry granule composition comprising
compressing a pharmaceutical composition to 800 to 900 kPa hardness to produce one or more slugs, and
milling the one or more slugs with an oscillating granulator to form granules.

2. The method of claim 1 further comprising sizing the granules.

3. The method of claim 2 wherein said sizing is performed with the oscillating granulator.

4. The method of claim 1 wherein the oscillating granulator has a 0.25 inch screen for milling the slugs.

5. The method of claim 3 wherein the oscillating granulator has a 16 mesh screen for sizing the granules.

6. The method of claim 2 wherein the sized granules have an average diameter from about 100 microns to about 200 microns.

7. The method of claim 6 wherein the sized granules have an average diameter of about 150 microns.

8. The method of claim 2 wherein no more than 35% of the sized granules have a diameter that is about 75 microns or less.
